(19) United States
(12) Patent Application Publication
(10) Pub. No.: US 2021/0238683 A1
(43) Pub. Date: Aug. 5, 2021

(54) Compositions and Methods for Diagnosis and Treatment of Endometriosis

(71) Applicant: The Board of Regents of the University of Texas System, Austin, TX (US)

(72) Inventors: Nameer KIRMA, San Antonio, TX (US); Bruce NICHOLSON, San Antonio, TX (US)

(21) Appl. No.: 17/048,953

(22) PCT Filed: Apr. 22, 2019
(86) PCT No.: PCT/US2019/028478

§ 371 Date: Oct. 19, 2020

Related U.S. Application Data
(60) Provisional application No. 62/660,641, filed on Apr. 20, 2018.

(51) Int. Cl. C12Q 1/6883; G01N 33/569

(52) U.S. Cl. CPC C12Q 1/6883; G01N 2800/364; C12Q 2600/158; G01N 33/56966

(57) ABSTRACT

Methods for the detection and diagnosis of endometriosis are disclosed herein. More specifically, sensitive single-cell expression analysis methods to detect specific gene expression patterns in cell populations obtained from endometrial cells are provided.

### CROSS-REFERENCE TO RELATED APPLICATIONS

This application claims benefit of and priority to U.S. Provisional Patent Application No. 62/660,641 filed on Apr. 20, 2018, which is incorporated by reference in its entirety.

### TECHNICAL FIELD OF THE INVENTION

The invention is generally directed to methods of detecting and diagnosing endometriosis.

### BACKGROUND OF THE INVENTION

Endometriosis is one of the most common gynecological diseases in the United States. It is a painful, often debilitating disease that affects more than 6.5 million women in America between the ages of 15 and 44 (Buck Louis, et al., Fertil Steril, 96:360-365 (2011)). Endometriosis occurs when the lining of the uterus grows ectopically, most commonly on the ovaries, fallopian tubes, tissues that hold the uterus in place, and the outer surface of the uterus. While less common, endometrial growths can also be found on the vagina, cervix, vulva, bowel, bladder, or rectum. The most common symptoms of endometriosis include pain, bleeding or spotting, infertility, and digestive problems such as diarrhea, constipation, bloating, or nausea.

The cause of endometriosis is unknown. Possible causes include retrograde menstrual bleeding, genetic factors, immune system problems, hormonal imbalance, and surgical complications. Because the cause of endometriosis is not known, current treatments for endometriosis only treat the symptoms, not the disease itself. In addition, there is no cure for endometriosis aside from surgery to remove the endometriotic lesions. Removal of endometriotic lesions is not a permanent cure for the disease it is merely a temporary therapeutic option.

Endometriosis is difficult to diagnose because its symptoms overlap with many other diseases affecting the abdomen and bowel. Currently, diagnosis of endometriosis relies on a combination of clinical history and both invasive and non-invasive tests. Pelvic exam, ultrasound, and MRI are all techniques used to visualize potential endometriotic lesions. However, a definitive diagnosis of endometriosis can currently only be obtained through a type of surgery called laparoscopy, wherein a doctor can look directly at the endometrial tissue from within the pelvic area. During a laparoscopy the doctor will also resect any endometriotic lesions that are found. Because laparoscopy is an invasive method, and it is associated with a high cost, screening for endometriosis is not practical. As a result, many women with endometriosis go undiagnosed, or incorrectly diagnosed, and untreated. There is a need for less invasive, earlier detection methods for diagnosing endometriosis.

Therefore, it is an object of this invention to provide more effective methods for diagnosing endometriosis.

It is also an object of the invention to provide a less invasive method for diagnosing endometriosis.

### SUMMARY OF THE INVENTION

Methods of diagnosing and treating endometriosis in patients are disclosed herein. Current methods of diagnosing endometriosis are either low-efficiency or are highly invasive. Efficient, non-invasive methods of diagnosing endometriosis are provided.

One embodiment provides a method of diagnosing and treating endometriosis in a subject in need thereof by enriching or expanding endometrial stromal cells obtained from the subject, subjecting the enriched or expanded endometrial stromal cells to single-cell processing in microfluidic chambers to produce amplified cDNA, subjecting the amplified cDNA to microfluidic PCR to detect RNA gene expression of one or more genes selected from the group consisting of DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, ZO2, and combinations thereof, diagnosing the subject with endometriosis if expression of the one or more genes is reduced relative to expression of the one or more genes in endometrial stromal cells from a subject without endometriosis, and administering a treatment for endometriosis to the subject diagnosed with endometriosis.

Another embodiment provides a method of diagnosing and treating endometriosis in a subject in need thereof by enriching or expanding endometrial epithelial cells obtained from the subject, subjecting the enriched or expanded endometrial epithelial cells to single-cell processing in microfluidic chambers to produce amplified cDNA, subjecting the amplified cDNA to microfluidic PCR to detect RNA gene expression of one or more genes selected from the group consisting of DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, ZO2, and combinations thereof, diagnosing the subject with endometriosis if expression of the one or more genes is elevated relative to expression of the one or more genes in endometrial epithelial cells from a subject without endometriosis, and administering a treatment for endometriosis to the subject diagnosed with endometriosis.

Yet another embodiment provides a method of diagnosing and treating endometriosis in a subject in need thereof by enriching or expanding endometrial stromal and epithelial cells obtained from the subject, subjecting the enriched or expanded endometrial stromal and epithelial cells to single-cell processing in microfluidic chambers to produce amplified cDNA, subjecting the amplified cDNA to microfluidic PCR to detect RNA gene expression of one or more genes selected from the group consisting of DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, ZO2, and combinations thereof, diagnosing the subject with endometriosis if expression of the one or more genes is reduced relative to expression of the one or more genes in endometrial stromal cells from a subject without endometriosis and elevated relative to expression of the one or more genes in endometrial epithelial cells from a subject without endometriosis, and administering a treatment for endometriosis to the subject diagnosed with endometriosis.

The endometrial cells can be obtained from menstrual blood or endometrial biopsy. In one embodiment, the stromal cells are isolated by sorting the cells using endometrial stromal cell markers CD10, CD146, and CD13. In another embodiment, the endometrial epithelial cells are isolated by soring the cells using endothelial epithelial cell markers EpCam+, CD45, and CD9.

The treatment for endometriosis can be selected from the group comprising anti-inflammatory drugs, hormonal therapy, or surgical removal of the affected tissue.

In one embodiment, the subject has symptoms of endometriosis. In another embodiment, the subject has been previously diagnosed with endometriosis.

### BRIEF DESCRIPTION OF THE DRAWINGS

FIGS. 1A-1I show heat maps reflecting levels of gene expression for gap junction genes (connexins) as well as other proteins involved in cell-cell interactions (tight and adhesion junctions), and various kinases that regulate them. In black and white, highest expression is grey, intermediate expression is black and lowest expression is white. Each dot corresponds to the expression of a particular gene (arrayed in rows) in a particular cell (arrayed in columns). FIGS. 1A-1E are gene expression profiles of stromal cells enriched from uterine endometrial biopsy samples from women without endometriosis (normal; FIG. 1A), early endometriosis (stage I/II; FIG. 1B), and more extensive endometriosis lesions in the pelvic cavity (stage III/IV; FIGS. 1C-1E). These samples were obtained at different stages of the menstrual cycle: ES, early secretory (FIGS. 1A-1C); MS, mid secretory (FIG. 1D); and P, proliferative (FIG. 1E). FIGS. 1F-1I are gene expression profiles of epithelial cells enriched from uterine endometrial biopsy samples from women without endometriosis (normal; FIGS. 1F-G), early endometriosis (stage I/II; FIG. 1H), and extensive endometriosis (stage III/IV; FIG. 1I). These samples were obtained at different stages of the menstrual cycle: ES, early secretory (FIGS. 1F-H) and P, proliferative (FIG. 10.

FIGS. 2A-2BB are box plots of single cell microfluidic PCR expression data for most gap junction genes (indicated by GJ above each plot) in enriched stromal cells (FIGS. 2A-2N) and epithelial cells (FIGS. 2O-2BB) from brush uterine biposies. Patients are identified by number along the X axis of each plot, and represent normal, early and late stage endometriosis subjects, as indicated in Table 3 of the Examples. The Y axis represents Log 10 expression. GJA1 (encoding the Cx43 protein) is the most abundantly expressed connexin in both cell types. However, virtually all connexins show a consistent and significant (indicated by asterisks) decrease in expression with disease progression in Stromal cells (FIGS. 2A-2N), but an increase with disease progression in epithelial cells (FIGS. 2O-2BB).

FIGS. 3A-3N are box plot showing single-cell microfluidic PCR expression data for several other genes involved in cell-cell interactions other than gap junctions and their regulators, as indicated at the top of each plot (TJP1=ZO1; Cav=caveolin; CDH2=N-cadherin; Vim=vimentin; CTNNB=catenin beta; PRHACA—Protein Kinase A; PRKCB=protein kinase C) in enriched endometrial stromal cells. (FIGS. 3A-3G) and endometrial epithelial cells (FIGS. 3H-3N) from brush biopsies of the uterus. Labeling of graphs is as in FIGS. 2A-2BB.

FIGS. 4A-4G show the functional assessment of gap junction intercellular coupling in primary endometrial stromal and epithelial cells from normal and endometriosis subjects. FIG. 4A is a schematic that shows how the assay is performed by loading “donor” cells with a gap junction permeant dye, dropping them onto a monolayer of recipient cells and following spread of the dye to the monolayer. FIGS. 4B-4E are representative images showing examples of the assay using stromal cells from normal subjects and an advanced endometriosis patient. FIG. 4F is a line graph showing the rate of transfer of calcein between donor and recipient cells over time (upper graph—normal; lower graph—endometriosis. The X-axis represents time and the Y-axis represents number of recipients labeled per donor cell. The slope is used as a measure of intercellular coupling efficiency (referred to as “coupling”). FIGS. 4G-4H are bar graphs showing coupling levels of epithelial endometrial cells (FIG. 4G) and stromal endometrial cells (FIG. 4H) to either one another (homotypic coupling—black bars) or to a monolayer of LP9 peritoneal mesothelial cells (heterotypic coupling—grey bars). Firstly, as expected from the expression profiles, stromal cells are less well coupled to one another than epithelial cells. However, when exposed to mesothelial cells (through which they normally invade in endometriosis) a dramatic increase in coupling is seen, but ONLY in stromal cells from endometriosis patients (significance is shown from a paired T-test).

FIGS. 5A-5F are fluorescence microscopy images showing expression of Cx43 in stromal cells alone (FIG. 5A-FIG. 5C) or stromal cells exposed to mesothelial cells (FIGS. 5D-5F) from uterine endometrial biopsy samples from women without endometriosis (167 normal; FIG. 5A, FIG. 5D), superficial endometriosis (164 Endo I-II; FIG. 5B, FIG. 5E), and deep infiltrating endometriosis (169 Endo III-IV; FIG. 5C, FIG. 5F). Arrows show redistribution of Cx43 to the cell surface in the endometriosis cells exposed to mesothelial cells, potentially explaining the rapid induction of coupling seen in FIGS. 4A-4H.

FIG. 6 is a bar graph showing the number of endometrial epithelial cells and stromal cells that invaded through a mesothelial cell monolayer in untreated samples (black bars) or samples treated with Cx43 siRNA to reduce cell coupling (grey bars) or control siRNA to glutaraldehyde dehydrogenase (GAPDH—hatched bars). Both cell types are invasive, and this invasiveness is largely eliminated when Cx43 expression is suppressed. However, stromal cells show a significant increase in invasiveness with disease progression, and this is dependent on Cx43 expression ONLY in endometriosis samples.

### DETAILED DESCRIPTION OF THE INVENTION

### Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “uterus” refers to an organ of the female reproductive system, also known as the womb. The main function of the uterus is to house and nourish a fetus until its ready for birth.

As used herein, “endometrium” refers to the mucous membrane lining the uterus. The endometrium changes throughout the menstrual cycle. Menstruation is the cyclic, sloughing of the endometrium in response to hormonal fluctuations. The menstrual cycle is divided into two phases, the follicular or proliferative phase, and the luteal or secretory phase. The proliferative phase is characterized by the development of ovarian follicles. The secretory phase is typically 14 days long and begins after ovulation.

As used herein, “endometrial cells” refer to cells from the endometrium. Endometrial cells can be subdivided into stromal cells and epithelial cells.

As used herein, “stromal cells” refer to connective tissue cells of any organs. Stromal cells support the function of the parenchymal cells of that organ. Endometrial stromal cells are important in the initiation and maintenance of pregnancy.

As used herein, “epithelial cells” refer to cells that form cohesive sheets of cells referred to as epithelia. They function as a covering or lining for body surfaces, and as functional units of secretory glands. Epithelial cells can be specialized for absorption, secretion, or to act as a barrier.

As used herein, “endometriosis” refers to a gynecological disease wherein tissue from the uterus grows in the abdominal cavity, outside of the uterus. The two main symptoms of endometriosis include pain and infertility. The main causes of the pain and infertility are endometrial implants and adhesions. As used herein, “endometrial implants” refer to endometrial tissue found in ectopic locations. Implants resemble small, flat patches on the peritoneal surface of the pelvic region. These implants cause irritation and inflammation in the surrounding tissue, which can lead to the formation of adhesions. “Adhesions” as used herein refer to bands of internal scar tissue that can bind tissues and organs that are normally mobile.

Endometriosis is classified in “stages” based on the severity of the disease, the extent of spread of the disease, the involvement of pelvic structures, the extent of pelvic adhesions, and blockage of the fallopian tubes. Staging of endometriosis does not necessarily reflect the severity of symptoms experienced by the patient.

The four stages of endometriosis are equivalent to minimal, mild, moderate, and severe. The majority of patients fall within minimal and mild stages. Minimal endometriosis, called Stage I, is characterized by isolated implants and no significant adhesions. Mild endometriosis, Stage II, is characterized by superficial implants less than 5 cm in aggregate without significant adhesions. Stage I and Stage II endometriosis are often combined in the same category called “superficial endometriosis”. Moderate (Stage III) endometriosis is characterized by the appearance of endometriomas, which are a type of cyst that forms when endometrial tissue grows in the ovaries. Severe endometriosis (Stage IV) is characterized by multiple implants, cysts, and severe adhesions which lead to scarring around the tubes and ovaries. Women with stage IV endometriosis are the most likely to have infertility problems.

As used herein, “gap junction” refers to an organized aggregate of protein channels in cell membranes that allow ions and small molecules to pass between adjacent cells.

### II. Methods of Diagnosing and Treating Endometriosis

Methods for diagnosing and treating endometriosis are provided herein. Disclosed herein are sensitive single-cell expression analysis methods to detect specific gene expression patterns in cell populations obtained from endometrial cells. An exemplary method includes steps of enriching or expanding endometrial cells obtained from the subject, subjecting the cells to single-cell processing in microfluidic chambers to produce amplified cDNA, subjecting the amplified cDNA to microfluidic PCR to detect RNA gene expression of one or more genes selected from the group containing DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, ZO2, and combinations thereof, diagnosing the subject with endometriosis if expression of one or more genes is elevated relative to expression of one or more genes in uterine stromal cells from a subject without endometriosis, and administering a treatment for endometriosis to the subject diagnosed with endometriosis.

A. Enrichment and Expansion of Endometrial Cells

1. Endometrial Samples

In one embodiment, samples of uterine cells are collected from women during routine wellness checks. In some embodiments, the women are suspected of having endometriosis. In another embodiment, the women have been previously diagnosed with endometriosis and the disclosed methods are used to monitor disease recurrence.

In one embodiment, the endometrial cells are obtained from the subject in a non-invasive manner, such as in menstrual fluid collected in stericups. In another embodiment, the endometrial cells are obtained from the subject in a minimally invasive method, such as with an endometrial brush biopsy device or endometrial suction catheter.

The women with endometriosis can have superficial endometriosis (stage I/II) or deep infiltrating endometriosis (stage III/IV). The biopsy specimen can be obtained at different stages of the menstrual cycle including but not limited to early secretory, mid secretory, or proliferative stages.

Some embodiments provide a method of subjecting uterine cells to single-cell processing and microfluidic PCR. The uterine cells can be stromal or epithelial.

The endometrial biopsies can be prepared for enrichment, expansion, and single-cell processing by isolating cells from the tissue. Methods for isolating cells from endometrial biopsies are known in the art. Exemplary methods include, but are not limited to, collagenase digestion, trypsin digestion, and manual scraping of surface epithelium from the whole biopsy (Krjutškov, K., et al. Human Reproduction, 31:844-853 (2016); Jividen, K., et al., J Vis Exp, 87:e51513).

The menstrual fluid is prepared for enrichment, expansion, and single-cell processing by removing red blood cells and isolating endometrial cells from the fluid.

2. Cell Enrichment

In one embodiment the population of endometrial cells from the biopsy is enriched for stromal cells or epithelial cells. Methods of enriching endometrial cell populations for stromal or epithelial cells are known in the art. Exemplary methods include but are not limited to physical separation using filtration devices, flow cytometry, magnetic beads or microbeads coated with specific antibodies, and microfluidics. The enriched samples can be expanded in culture prior to single-cell processing.

i. Filtration

In one embodiment, stromal cells are isolated from the endometrial cell population by passing the digested cell suspension through a cell culture filter. Stromal cells will flow through the filter while epithelial cells will remain aggregated within the tissue that is not passed through the filter. The epithelial tissue can be further digested into a single cell suspension. The enriched cell suspensions can be cultured for expansion or used directly for single-cell processing.

ii. FACS

Fluorescence Activated Cell Sorting (FACS) is a specialized type of flow cytometry with sorting capacity that can isolate single cells. Before separation, a cell suspension is made and the target cells are labeled with fluorescent probes. Fluorophore-conjugated monoclonal antibodies (mAb) are the most widely used fluorescent probes that recognize specific surface markers on target cells. As the cell suspension runs through the machine, each cell is exposed to a laser, which allows the fluorescence detectors to identify cells based on the selected characteristics, particularly which antibodies are bound. The instrument applies a charge (positive or negative) to the droplet containing a cell of interest and an electrostatic deflection system facilitates the collection of the charged droplets into appropriate collection tubes for later analysis. FACS can also be used to sort single cells.

In one embodiment, the cells from the endometrial biopsy are sorted using FACS. The cell suspension from the endometrial biopsy can be incubated with fluorescent probes that recognize stromal cell markers such as CD10, CD146, and CD13. The cell suspension is run through the flow cytometer and the stromal cells are collected in a separate container. In another embodiment, the cell suspension is incubated with fluorescent probes that recognize epithelial markers such as EpCam+ and CD9. The cell suspension is run through a flow cytometer and the epithelial cells are collected in a separate container. In one embodiment, the endometrial cell culture is enriched for stromal or epithelial cells using FACS.

iii. Magnetic Beads

Magnetic bead cell isolation is a technique used to enrich a specific cell type from a mixed population of cells. Magnetic beads or nanoparticles conjugated with antibodies against cell surface markers on the target cell are mixed with the population of cells. The container holding the cells is exposed to a magnetic column and the cells of interest (which are conjugated with the magnetic beads) are separated from the remainder of the cells. In one embodiment, the magnetic beads are conjugated with antibodies against stromal cell markers such as CD10, CD146, and CD13 and are used to separate the stromal cells from the endometrial cell suspension.

iv. Microfluidics

In another embodiment, the cells from the endometrial biopsy are enhanced using microfluidics. Cell sorting by a microfluidic chip can be divided into four categories: cell-affinity chromatography based microfluidic separation, physical characteristics of cell based microfluidic separation, immunomagnetic beads based microfluidic separation, and separation methods based on differences between dielectric properties of various cell types.

Cell-affinity chromatography based microfluidic is the most commonly used method for microfluidic chip analysis. It is based upon highly specific interactions between antigen and antibody, ligand and receptor. At the beginning of the process, the micro-channel in the chip is modified with specific antibodies capable of binding to cell surface antigen or aptamer. Once the sample flows through the micro-channels, its cell surface antigen can bind to the specific antibodies or aptamer immobilizing the cells on the chip, while the remaining cells flow off the chip with the buffer. Finally, using a different buffer, the immobilized cells can be eluted for downstream analysis. In one embodiment, the endometrial cell population is enriched for stromal cells using microfluidics. In another embodiment, epithelial cells are enriched.

3. Cell Expansion

In other embodiments the cells are expanded in culture without being enriched for specific subtypes of cells. The endometrial biopsies can be prepared for expansion in cell culture by isolating cells from the tissue. Methods for isolating cells from endometrial biopsies are known in the art. Exemplary methods include but are not limited to collagenase digestion, trypsin digestion, and manual scraping of surface epithelium from the whole biopsy (Krjutškov, K., et al. Human Reproduction, 31:844-853 (2016); Jividen, K., et al., J Vis Exp, 87:e51513). Methods for culturing endometrial cells are known in the art. See for example Osteen, K. G., et al., Fertility and Sterility, 52:966-972 (1989); Zhang, L., et al., J Cell Sci, 108:323-331(1995). In some embodiments, the high sensitivity of the single-cell PCR overcomes the need to expand cells in culture before analysis.

B. Single-Cell Processing

In one embodiment, the enriched or expanded stromal or uterine cells are subjected to single-cell processing. Single-cell processing is the method of isolating a single cell from a population of cells and performing analysis on the single cell rather than whole populations of cells. In one embodiment, the single cells from the endometrial samples are subjected to microfluidic PCR.

1. Single Cell Isolation

In one embodiment, stromal cells are isolated from the endometrial cell culture. Exemplary endometrial stromal cell markers include but are not limited to CD10, CD146, and CD13. In another embodiment, epithelial cells are isolated from the endometrial cell culture. Exemplary epithelial markers include but are not limited to EpCam+, CD45, and CD9.

Methods for isolating single cells from a total cell culture are known in the art. Exemplary methods for isolating single cells from large populations of cells include manual cell picking, flow cytometry, magnetic-activated cell sorting, and microfluidics.

Methods for isolating rare cells from a population, or isolating single cells from a small sample are also known in the art. Dielectropheretic (DEP) microfluidic systems use a microfluidics chip with dielectropheretic cages to navigate individual cells by charge after identification with fluorescent markers. The advantage of these systems is that every cell is preserved, and even a single cell in a pool of 100,000 can be isolated efficiently. An exemplary DEP system is the DEP-Array™ system (Silicon Biosciences). In one embodiment, the endometrial cells are separated into single cell samples using a DEP system. The cells can be labeled with stromal or epithelial markers to separate the two populations of cells. The DEP system can distribute the single cells each into their own individual well of a microplate for further processing.

2. Microfluidic PCR

In one embodiment, the cDNA from the processed single-cells is used for microfluidic PCR. Microfluidics are microminiaturized devices that can process samples with volumes of fluid on the order of nanoliters or picoliters. Microfluidic PCR systems can successfully detect nucleic acid expression from nanoliter sized samples. In one embodiment the microfluidic PCR machine is a single-cell microfluidic PCR machine. An example of microfluidic PCR machines on the market are the BioMark™ HD single-cell system (Fluidigm) or the C1™ system (Fluidigm).

i. Gap Junctions

Gap junctions are specialized intercellular connections between cells. These connections, or channels, provide direct intercellular communication between the cytoplasm of two cells allowing rapid exchange of ions and metabolites up to approximately 1 kD in size. Gap junctions are formed from clusters of connexin proteins. Exemplary gap junction genes include but are not limited to GJA1, GJA3, GJA5, GJA8, GJA9, GJB1, GJB2, GJB3, GJB4, GJB5, GJB6, GJB7, and GJC2. In one embodiment, expression of gap junction genes is elevated in endometrial epithelial cells, but reduced in endometrial stromal cells from women with endometriosis.

Intercellular gap junction communication has been examined as a mode of cellular communication between endometrial cells and the target of invasion in endometriosis, the mesothelium. Using traditional cell assays as well as single-cell analysis, specific gap junction proteins (channel forming connexins) were identified that may be involved in endometrial cell invasiveness leading to endometriotic lesion development. In addition to the gap junction genes, other regulatory genes and kinases are involved in communication at gap junctions. Exemplary kinases that regulate gap junctions include, but are not limited to, tyrosine kinases, protein kinase C, cAMP-dependent protein kinase, MAP kinases, cdc2/cyclinB, and casein kinase I (Warn-Cramer, B. J. and Lau, A. F., Biochim Biophys Acta, 1662:81-95 (2004); Lampe, P. D. and Lau, A. F., Int J Biochem Cell Biol, 36:1171-1186 (2004)).

In one embodiment, the RNA gene expression of DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, and ZO2 in the cDNA from single-cell samples are measured by microfluidic PCR.

In another embodiment, the expression level of one of the disclosed genes in a sample is compared to the expression level of that same gene in a sample from a healthy individual without endometriosis.

C. Endometriosis Diagnosis

In the disclosed methods, a subject is diagnosed with endometriosis if the expression of one or more genes from the group containing DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, and ZO2 are elevated relative to expression of one or more of the genes in endometrial epithelial cells from a subject without endometriosis or reduced relative to expression of one or more genes in endometrial stromal cells from a subject without endometriosis.

The expression levels of the above-mentioned genes increase or decrease progressively with disease severity. Therefore, in one embodiment the expression level of the gene can be used to determine the stage of endometriosis. Among the genes with most differential expression between endometriosis and normal endometrial epithelial cell samples are CDH2, Vimentin and CTNNB, as in examples (Table 1, below).

TABLE 1
Expression of various genes in endometrial epithelial cells.
Log10 expression*:

| | | | | | | |
|---|---|---|---|---|---|---|
| Sample | Range | Max | Min | Median | 25% | 75% |

CDH2

| | | | | | | |
|---|---|---|---|---|---|---|
| 172 | 2.923 | −0.554 | −3.477 | −2.527 | −2.904 | −2.265 |
| 164 | 2.331 | 0.119 | −2.212 | −1.389 | −1.685 | −1.222 |
| 170 | 9.739 | 0.906 | −8.834 | −1.439 | −1.667 | −0.629 |

Vimentin

| | | | | | | |
|---|---|---|---|---|---|---|
| 172 | 1.133 | 0.0835 | −1.049 | −0.355 | −0.575 | −0.247 |
| 164 | 0.998 | 0.586 | −0.413 | −0.0282 | −0.160 | 0.125 |
| 170 | 1.217 | 1.016 | −0.201 | 0.206 | 0.0115 | 0.347 |

CTNNB

| | | | | | | |
|---|---|---|---|---|---|---|
| 172 | 1.044 | −0.713 | −1.757 | −1.335 | −1.448 | −1.149 |
| 164 | 1.912 | 0.563 | −1.350 | −0.997 | −1.143 | −0.847 |
| 170 | 2.836 | 1.536 | −1.301 | −0.844 | −1.068 | −0.331 |

172: normal
164 endometriosis stage I/II
172: endometriosis stage III/IV
*Expression is defined by 2^{-ΔCt}, where Ct is the PCR cycle threshold of each gene and Δ (Greek, delta) is the difference between the target gene and a normalizing housekeeping gene (GAPDH).

In one embodiment, the woman being tested for endometriosis has symptoms of endometriosis or has a family history of the disease. The woman being tested for endometriosis can be going through fertility treatment or suffering from infertility. In one embodiment, the woman being tested for endometriosis is of reproductive age. The woman could be between the age of 15 and 45.

D. Endometriosis Therapeutics

In one embodiment, the subject is diagnosed with endometriosis based on expression levels of the disclosed genes and is subsequently treated for endometriosis. Treatments for endometriosis are directed toward alleviation of the symptoms of the disease. The most common symptoms of the disease include pain, bleeding, and infertility.

i. Pain Relievers

One embodiment provides treatments for endometriosis induced pain. Pain medication can be used for mild pain and inflammatory symptoms of endometriosis. The most common type of pain reliever is non-steroidal anti-inflammatory drugs (NSAID). Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Steroidal anti-inflammatory agents can also be used to treat pain. Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

If pain is so severe that anti-inflammatory drugs are not effective, women can be prescribed narcotic pain relievers such as but not limited to morphine, fentanyl, oxycodone, tramadol, hydromorphone, and codeine.

ii. Hormone Therapeutics

Another treatment for endometriosis-associated pain and bleeding is hormone therapeutics. Because the ectopic endometrial tissue goes through a cycle similar to menstruation, hormones can sometimes be effective for treating pain associated with the disease. Hormones can be delivered in the form of a pill, a shot or injection, or a nasal spray. Oral contraceptives can be used to deliver hormones. Typically oral contraceptives contain two hormones, estrogen and progestin. Exemplary combination oral contraceptives include but are not limited to Desogestrel/ethinyl estradiol, Dienogest/estradiol valerate, Drospirenone/ethinyl estradiol, Drospirenone/ethinyl estradiol/levomefolate, Ethynodiol/ethinyl estradiol, Levonorgestrel/ethinyl estradiol, Mestranol/norethindrone, Norethindrone/ethinyl estradiol, Norgestimate/ethinyl estradiol, and Norgestrel/ethinyl estradiol.

Progestins are a group of drugs that behave like the female hormone progesterone. Progestins can be taken as a pill, by injection, or through an intrauterine device (IUD). While the most commonly prescribed oral contraceptives are combination formulations of estrogen and progestin, progestin only contraceptives are also prescribed. They have been shown to improve symptoms of endometriosis by reducing a woman's period or stopping it completely.

Gonadotropin releasing hormone (GnRH) agonists are commonly prescribed to women with endometriosis. GnRH agonists come in different forms including three-monthly injection, monthly injection, daily injection, and nasal spray. Exemplary GnRH agonists include but are not limited to buserelin, goserelin, leuprorelin, leuprolide, naferelin, and triptorelin.

Danazol is an androgen that has been shown to be effective in the treatment of pelvic pain associated with endometriosis.

In one embodiment, women diagnosed with endometriosis using the disclosed methods are treated with hormonal therapeutics.

iii. Surgical Treatments

Surgery has been shown to provide significant pain relief from endometriosis. Laparoscopy is a type of surgery in which the surgeon makes a small incision in the abdomen and inserts a small viewing instrument called a laparoscope into the abdomen. This allows the surgeon to directly visualize the endometriotic lesions. The surgeon can make secondary incisions to insert lasers or other instruments into the abdomen to remove or destroy the lesions. In one embodiment, the patient that is diagnosed with endometriosis with the methods disclosed herein is treated by laparoscopy.

Laparotomy is a major abdominal surgery that can also be used to remove endometrial lesions. In this procedure the surgeon makes an incision across the abdomen to visualize the abdominal cavity and uterus. The surgeon can remove endometriosis lesions during a laparotomy. In one embodiment, the patient that is diagnosed with endometriosis with the methods disclosed herein is treated by laparotomy. The surgeon may also perform a hysterectomy during a laparotomy in which the entire uterus is removed. Patients with extreme pain or advanced or recurrent endometriosis can elect to have a hysterectomy to eradicate endometriosis. In one embodiment, the patient that is diagnosed with endometriosis with the methods disclosed herein is treated by hysterectomy.

Women having pain in the center of their abdomen can have nerves in their pelvis severed to lessen the pain. This can be done via laparoscopy or laparotomy. There are two procedures that sever different nerve pathways. Presacral neurectomy severs the nerves connected to the uterus. In one embodiment, the patient that is diagnosed with endometriosis with the methods disclosed herein is treated by presacral neurectomy. The second procedure, called laparoscopic uterine nerve ablation, involves cutting nerves in the ligaments that secure the uterus. In one embodiment, the patient that is diagnosed with endometriosis using the methods disclosed herein is treated by laparoscopic uterine nerve ablation.

### EXAMPLES

### Example 1. Gap Junction Genes are Elevated in Endometriosis Derived Uterine Epithelial Cells and Reduced in Endometriosis Derived Stromal Cells

### Methods

Acquisition of Eutopic (from the Uterus) Endometrial Tissue from Women with and without Endometriosis.

The presence or absence of endometriosis was confirmed by laproscopy. All eutopic endometrial samples were obtained at the proliferative phase of the menstrual cycle from normally cycling reproductive age women (age range 30-45), who were not under hormonal medication. The endometriosis samples were obtained from patients classified as Stage I-IV based on the American Society of Reproductive Medicine classification. In the case of normal subjects, endometrial tissue was isolated from women undergoing tubal sterilization, who were not taking oral contraceptives and were demonstrated to be free of endometriosis by laparoscopy, with no evidence of submucosal myomas or endometrial polyps. Isolation of primary endometrial epithelial cells (EECs) and stromal cells (ESCs) from the biopsies was performed using the methods developed by Kirk and Irwin shown to achieve about 97% purity, which was confirmed by previous studies. Additionally, the epithelial nature of EECs was confirmed in this study using staining against the epithelial cell adhesion molecule (EpCAM) and expression of cytokeratin 18. ESCs were verified by vimentin staining. Table 2 shows the list of normal subject and patients used in the study. Numbers were limited due to the extensive analyses done on single cells, and the need to not have the primary cells passaged extensively prior to analysis.

TABLE 2
Patient base.

| | | | | | | | | | | | | | | | | |
|---|---|---|---|---|---|---|---|---|---|---|---|---|---|---|---|---|
| | | | | | | | | | | Prolif | ES | MS | LS | | | |
| Sample | | Patient | Patient | | | | Cycle | | 1-7 | 8-14 | 15-18 | 19-23 | 24- | pg/ml | ng/ml | Histology |
| # | Source | Ethnicity | Age | G | P | BMI | Length | CD | M | P | ES | MS | LS | E2 | P4 | read (Path) |

NORMAL CASES

| | | | | | | | | | | | | | |
|---|---|---|---|---|---|---|---|---|---|---|---|---|---|
| ME-167 | MAMC | Caucasian | 23 | 1 | 1 | 32.6 | 26 | 15 | X | | 94 | 9.8 | Secretory |
| ME-171 | MAMC | — | 35 | 0 | 0 | 27 | 32-35 | 28 | | X | 224 | 10.7 | Secretory |
| ME-172 | MAMC | Hispanic | 25 | 1 | 0 | 28.3 | 28-30 | 14 | X | | 148 | 6.1 | Early |
| | | | | | | | | | | | | | Secretory |

STAGE I-II ENDOMETRIOSIS CASES

| | | | | | | | | | | | |
|---|---|---|---|---|---|---|---|---|---|---|---|
| ME-164 | MAMC | Pacif. | 30 | 0 | 0 | 28 | 15 | X | 58 | 3.9 | Early |
| | | Islander | | | | | | | | | Secretory |

STAGE III-IV ENDOMETRIOSIS CASES

| | | | | | | | | | | | | | | |
|---|---|---|---|---|---|---|---|---|---|---|---|---|---|---|
| ME-163 | MAMC | Caucasian | 23 | 0 | 0 | 25 | 31-35 | 31 | | X | | 132 | 1.7 | Early |
| | | | | | | | | | | | | | | Secretory |
| ME-169 | MAMC | Caucasian | 37 | 0 | 0 | 23 | 28 | 20 | | | X | 168 | 26.8 | Secretory |
| ME-170 | MAMC | Caucasian | 30 | 0 | 0 | 22 | 28-30 | 10 | X | | | 92 | 0.1 | Prolif |

Cell Culture

Primary endometrial EECs and ESCs were cultured in Dulbecco's modified Eagle Medium (DMEM)/F12 (1:1) (Sigma, St Louis, Mo., USA) containing antibiotics and antimycotics, 5 μg/ml insulin (Sigma) and 10% fetal calf serum (Hyclone, Logan, Utah, USA) as described previously. Experiments were performed using low passages (<4). Established LP9 cells (Cornell Cell Repositories, Camden, N.J.) were used as a model for peritoneal mesothelial cells. For examining connexin 43 (Cx43) protein expression typical immunofluorescence staining was used for ESCs in culture, using green-fluorescence conjugated anti-Cx43 antibody. To experimentally suppress Cx43 expression in ESCs, siRNA specific to Cx43 was transfected into the cells.

Single-Cell RNA Screening of Connexin Gene Panel Expression by Microfluidic PCR

Primary EEC and ESC cultures were subjected to C1 (Fluidigm Corp) single cell isolation and processing to produce amplified cDNA from each cell. The cDNA was then subjected to microfluidic PCR gene expression analysis, using the Biomark platform (Fluidigm Corp). Corresponding PCR primer sequences connexin and gap junction regulator panel and were used for the detection of expression of these genes. In each microfluidic PCR chip assay, universal RNA (200 pg) from human normal tissues (cat #4234565, BioChain, Newark, Calif.) and no template control (NTC) served as positive and negative controls, respectively. Valid PCR products were determined by amplicon melting temperature curves for each gene amplicon.

Results

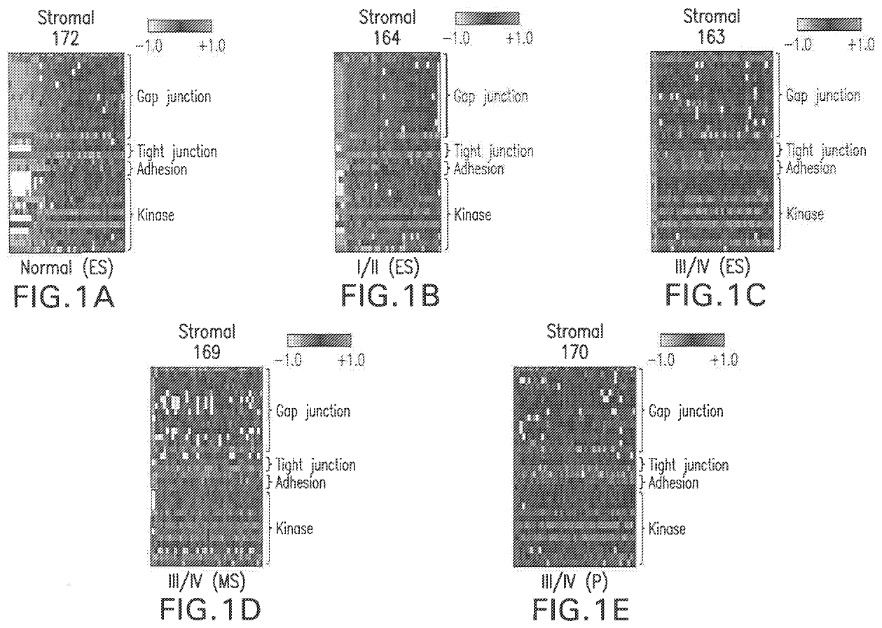

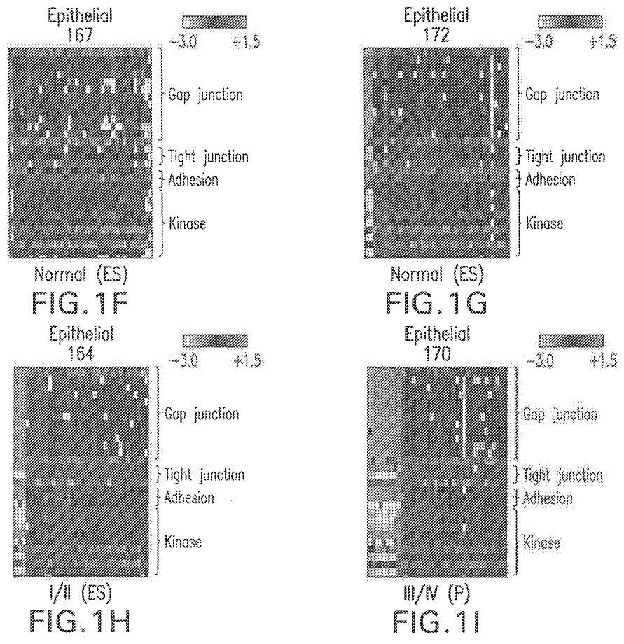

A pattern of decreased gap junction gene expression (upper portion of each plot in FIG. 1A-1I) was observed in the endometrial stromal cells from endometriosis patients when compared to those from healthy subjects, especially in stage III/IV endometriosis disease (FIGS. 1A-1E). Importantly, this was independent of the menstrual phase at which cells were harvested.

By contrast, a pattern of increased expression of many gap junction genes was observed in the enriched epithelial cells in endometriosis compared to normal derived samples (FIGS. 1F-1I). A progressive increase in gene expression is also observed with disease stage, but for several genes this was significant even at the earliest phases of endometriosis.

Changes were also seen in other genes involved in adhesive and sealing intercellular contacts and the kinases that regulate them (lower portion of each plot in FIG. 1A-10, but these were much less consistent compared to the Connexin patterns just mentioned.

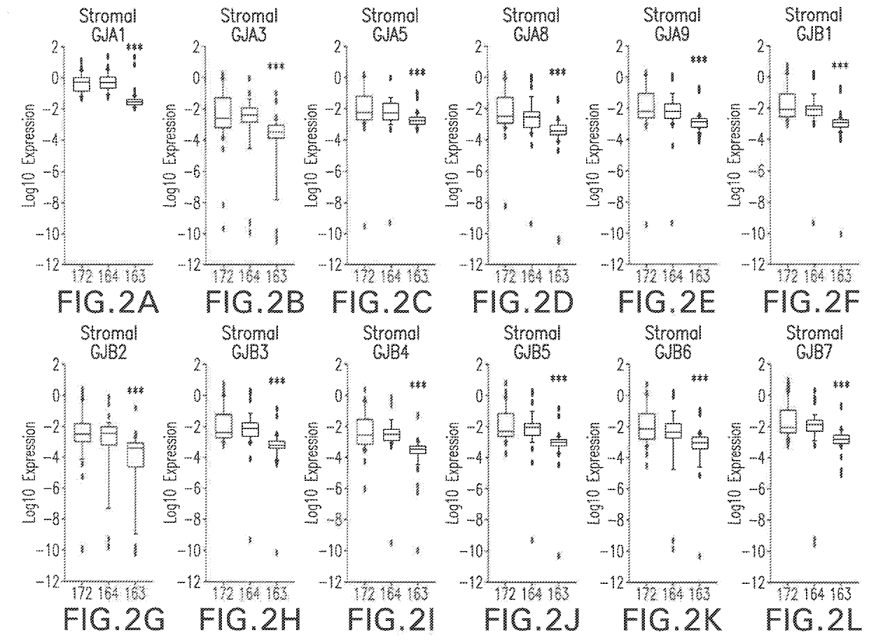

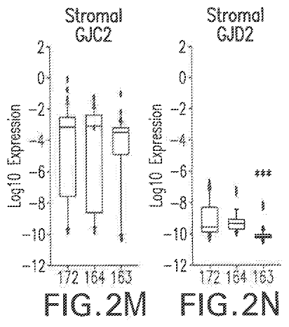

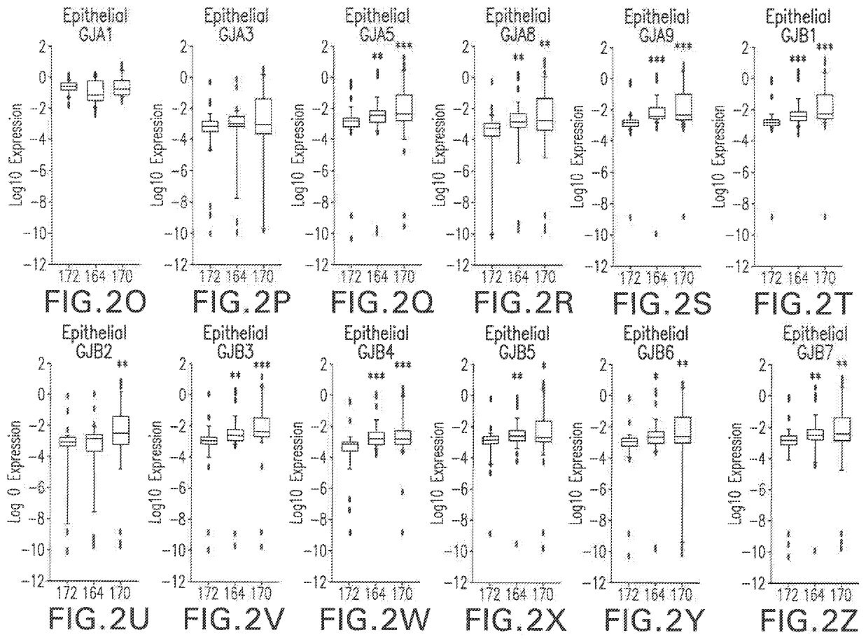

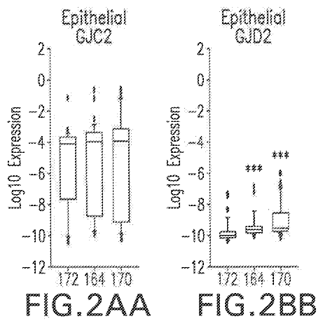

FIGS. 2A-2BB show quantitative PCR analysis of all connexin expression at the single cell level from a normal subject, and early and late stage endometriosis patients after separation of stromal (FIG. 2A-2N) and epithelial cells (FIG. 2O-2BB). Sample identifiers are described in Table 3. Virtually all connexin genes showed a progressive decrease in expression with disease progression in the stromal cells. The epithelial cells showed the opposite pattern, although the major connexin (GJA1) showed little difference between patients.

TABLE 3
Sample identifiers for FIGS. 2A-2BB and 3A-3N.

| Patient | Status | Stage | menstrual cycle |
|---|---|---|---|
| 172 | Normal | — | Early secretory |
| 164 | Endom | I/II | Early Secretory |
| 163 | Endom | III/IV | Early Secretory |
| 170 | Endom | III/IV | Proliferative |

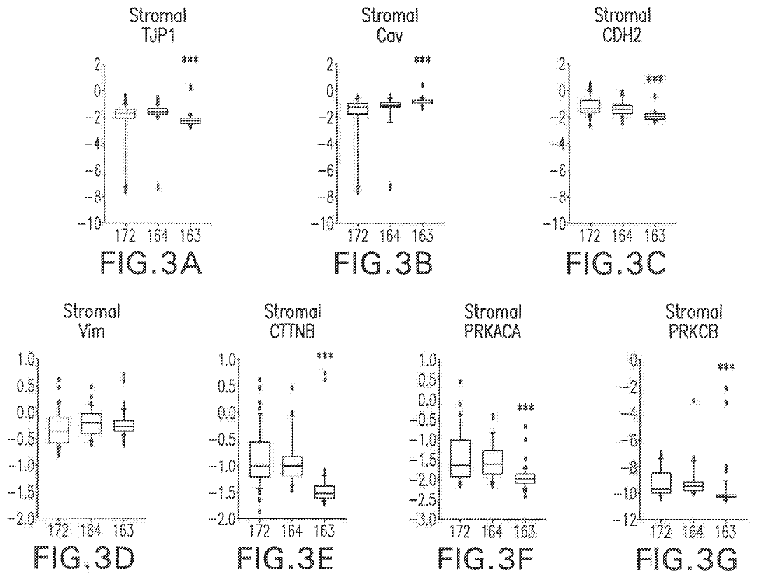

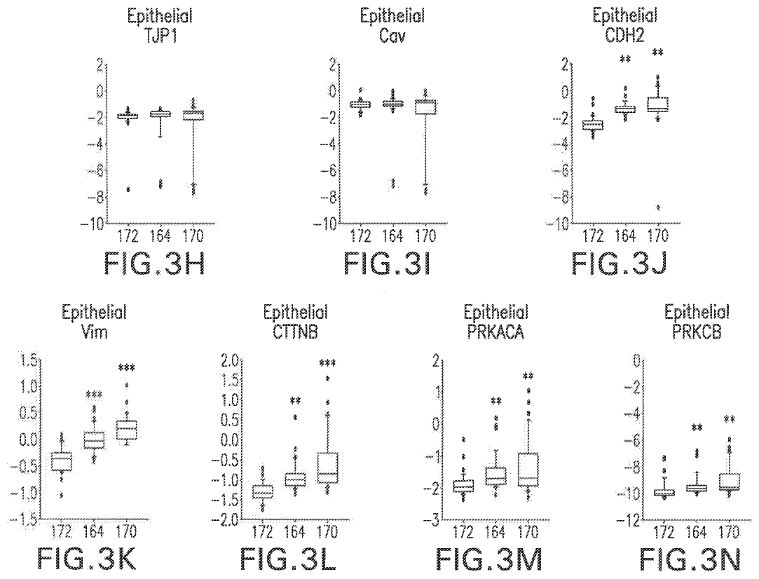

FIG. 3A-3N show a similar analysis of genes involved in the regulation of gap junction activity (PRKAC, PRKCB, Cav), or genes encoding cytoskeletal anchoring proteins (Vim) or other kinds of cell-cell interactions like adhesion (CDH2, CTNNB) and tight junctions (TJP1). While some showed no change with disease (TJP1, Cav in Epithelial cells, and others not shown), others showed expression patterns that mimicked the connexins (decreases with disease in stromal cells, but increases in epithelial cells).

### Example 2. Coupling of Endometrial Epithelial (E) and Stromal Cells (S) with Mesothelial Cells (M) and Invasiveness

Methods

Homotypic and Heterotypic Gap Junction Intercellular Communication (GJIC) Assays (Also Described as Coupling Assays)

GJIC was measured using intercellular transfer of the fluorescent dye Calcein, which is permeable via gap junctions. Assays were performed in culture media supplemented with charcoal-stripped FBS (10%). Donor cells were incubated with calcein AM for 20 minutes at room temperature. Inside the cell, calcein AM is cleaved by non-specific esterases into calcein, making it impermeable to diffusion through the cell membrane. Recipient cells are grown to confluence. Calcein-labeled donor cells were then dropped (‘parachuted’) onto the recipient cell layer, and calcein transfer between donor and recipient cells was observed with fluorescent microscopic imaging. For homotypic interactions, endometrial epithelial (EECs), endometrial stromal (ESCs) and mesothelial (LP9) donor cells were parachuted onto recipient cells of the same type, respectively. For heterotypic GJIC assays, EECs (or ESCs) were parachuted on LP9 recipient cells and vice versa. Initial optimization assays showed that dye transfer in EECs, ESCs and LP9 cells was optimally observed 1.5-2 hr after parachuting. Fluorescent images of 10-15 fields per well were captured on an Operetta automated microscope (Perkin Elmer). A program was written by Perkin Elmer that allowed identification of all cells on the plate, (from phase contrast image), as well as original donors (100±50 per well), and dye-filled recipients due to calcein transfer over time. Data are expressed as # of fluorescent recipient cells/# of donor cells for each condition.

Trans-Mesothelial Invasion Assay

The 3-D invasion assay modeling trans-mesothelial invasion was described previously. Briefly, LP9 peritoneal mesothelial cells (PMCs) were grown to confluence in 24-well invasion chamber inserts containing growth-factor-reduced Matrigel™, coated on 8-μm pore membranes (BD Bioscience, San Jose, Calif., USA). Then, 20,000 endometrial epithelial (EECs) or stromal (ESCs) cells were plated, after labeling with CellTracker Green® (Molecular Probes-Invitrogen, Carlsbad, Calif.) and treated with the relevant siRNA, on the confluent layer of LP9 PMCs in the prepared inserts. After 20 hr incubation, cells that did not invade, on the upper surface of the insert membranes, were mechanically removed. Invaded cells, on the bottom of the coated membranes, were stained with DAPI, were visualized using a fluorescence microscope with a 20× objective. Invasion assays for each cell type were performed in triplicates.

Results

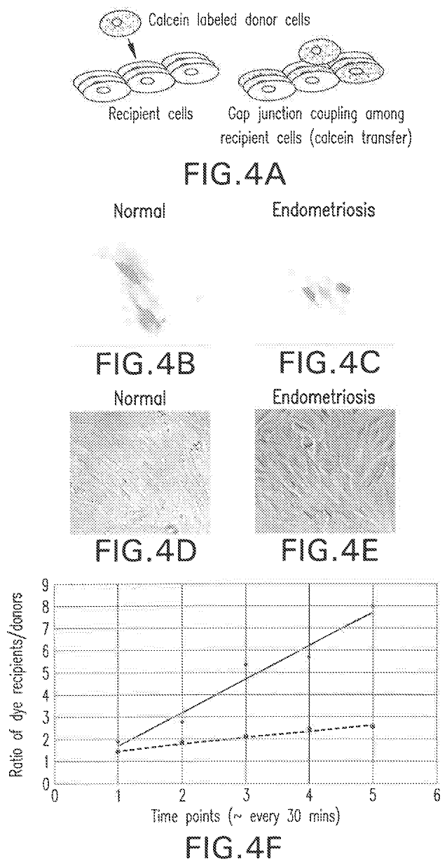

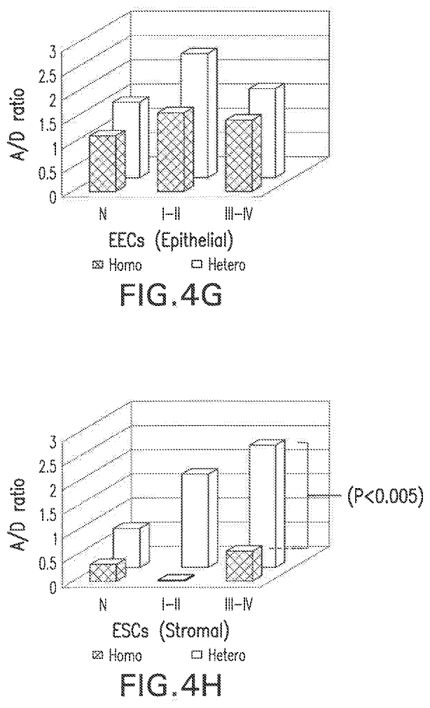

Coupling of cells was measured using a parachute assay where Calcein transfer between donor cells dropped onto a monolayer of recipient cells was measured over time (FIG. 4A-4F). This rate of transfer was determined for homotypic coupling between either epithelial cells (EECs—FIG. 4G) or stromal cells (ESCs—FIG. 4H) [black bars] and for heterotypic coupling of these cells with LP9 peritoneal mesothelial cells [grey bars] in normal (N), early stage (I-II) and late stage (III-IV) endometriosis patients. Mesothelial cells induced coupling in stromal cells from patients, but not from normal subjects (FIG. 4H), and not in epithelial cells from patients or normal subjects (FIG. 4G).

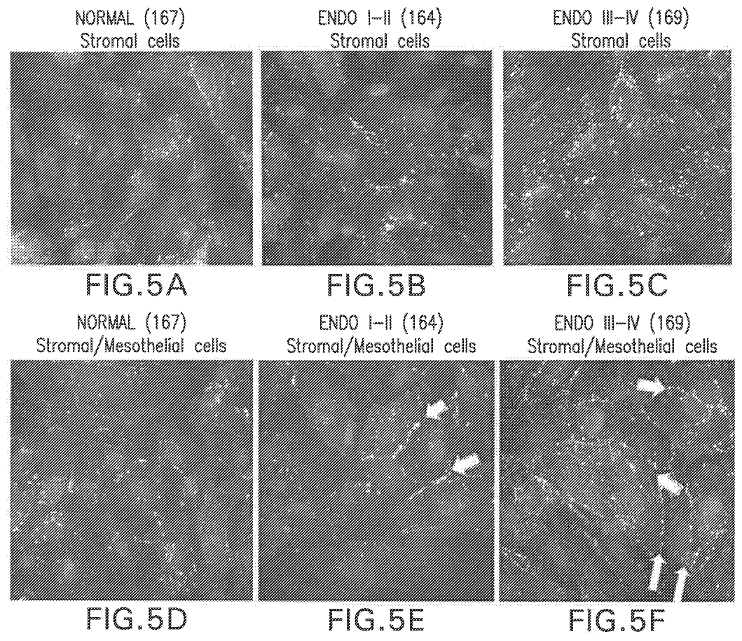

Immunofluorescent staining of Cx43 reveals an internal distribution of Cx43 in stromal cells from all subjects (note the staining does not concentrate around the edges of the cells and at cell-cell interfaces—FIGS. 5 A-C). This is more marked as endometriosis progresses, although surprisingly no noticeable reduction in expression is evident (FIG. 5A-5C). However, exposure of stromal cells to mesothelial cells causes a redistribution of Cx43 to the cell surface (arrows) in endometriosis samples (FIGS. 5E and 5F), but to a much lesser degree in cells from normal subjects (FIG. 5D). This activation of intracellular stores of Cx43 upon exposure to mesothelial cells could explain the dramatic and rapid increase in heterotypic coupling seen in FIGS. 4B-4E.

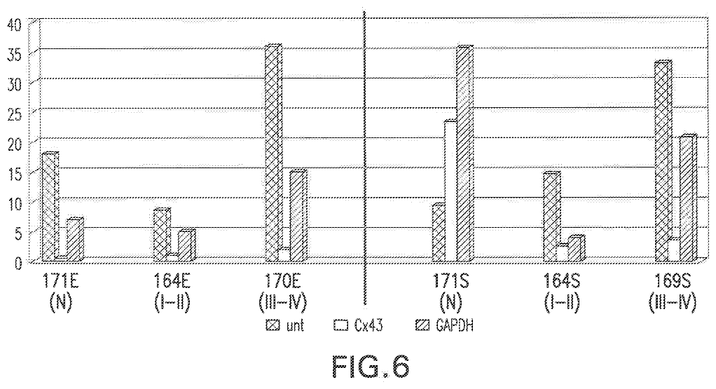

The invasion of endometrial epithelial cells (E) and stromal cells (S) through a mesothelial cell monolayer were also measured in a Boyden chamber to mimic the invasive process characteristic of endometriosis (FIG. 6). Endometrial cells were either left untreated [black bars] or treated with siRNA targeting Cx43 [grey bars] or a control protein, GAPDH [hatched bars]. Epithelial cells were invasive in a Cx43-depenmdent fashion, but this was variable between patients and did not correlate with disease state (FIG. 6). Stromal cells, in contrast showed increasing invasiveness with disease progression, but this was only dependent on Cx43 in the disease state.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A method of diagnosing and treating endometriosis in a subject in need thereof comprising
enriching or expanding endometrial stromal cells obtained from the subject;
subjecting the enriched or expanded endometrial stromal cells to single-cell processing in microfluidic chambers to produce amplified cDNA;
subjecting the amplified cDNA to microfluidic PCR to detect RNA gene expression of one or more genes selected from the group consisting of DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, ZO2, and combinations thereof; and
diagnosing the subject with endometriosis if expression of the one or more genes is reduced relative to expression of the one or more genes in endometrial stromal cells from a subject without endometriosis;
and administering a treatment for endometriosis to the subject diagnosed with endometriosis.

2. A method of diagnosing and treating endometriosis in a subject in need thereof comprising
enriching or expanding endometrial epithelial cells obtained from the subject;
subjecting the enriched or expanded endometrial epithelial cells to single-cell processing in microfluidic chambers to produce amplified cDNA;
subjecting the amplified cDNA to microfluidic PCR to detect RNA gene expression of one or more genes selected from the group consisting of DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, ZO2, and combinations thereof;
diagnosing the subject with endometriosis if expression of the one or more genes is elevated relative to expression of the one or more genes in endometrial epithelial cells from a subject without endometriosis;
and administering a treatment for endometriosis to the subject diagnosed with endometriosis.

3. A method of diagnosing and treating endometriosis in a subject in need thereof comprising
enriching or expanding endometrial stromal and epithelial cells obtained from the subject;
subjecting the enriched or expanded endometrial stromal and epithelial cells to single-cell processing in microfluidic chambers to produce amplified cDNA;
subjecting the amplified cDNA to microfluidic PCR to detect RNA gene expression of one or more genes selected from the group consisting of DBN1, CAV1, CDH, CDK1, CD45, CK19, CSNK, CTNNB1, Cx43, EpCAM, GAPDH, GJA1, GJA3, GJA5, GJA8, GJA9, GJB1, GJB2, GJB3, GJB4, GJB5, GJB6, GJB7, GJC2, GUSB, KRT18, MAPK1, MAPK3, MME, Notch1, NOV1, PECAM, PRKACA, PRKACB, PRKACG, PRKCA, SNAIL SRC, TGFBR2, TJAP1, TJP1, TJP2, Twist 1, VEGFR1, VIM, Zeb2, ZO1, ZO2, and combinations thereof;
diagnosing the subject with endometriosis if expression of the one or more genes is reduced relative to expression of the one or more genes in endometrial stromal cells from a subject without endometriosis and elevated relative to expression of the one or more genes in endometrial epithelial cells from a subject without endometriosis;
and administering a treatment for endometriosis to the subject diagnosed with endometriosis.

4. The method of claim 1, wherein the endometrial cells are obtained from menstrual blood.

5. The method of claim 1, wherein the endometrial cells are obtained from an endometrial biopsy.

6. The method of claim 1, wherein the treatment for endometriosis is selected from the group comprising anti-inflammatory drugs, hormonal therapy, or surgical removal of the affected tissue.

7. The method claim 1, wherein diagnosing the subject with endometriosis further comprises staging the endometriosis.

8. The method of claim 7, wherein the endometriosis is superficial endometriosis (stage I/II) or deep infiltrating endometriosis (stage III/IV).

9. The method of claim 1, wherein the subject has symptoms of endometriosis.

10. The method of claim 1, wherein the subject has been previously diagnosed with endometriosis.

11. The method of claim 10, wherein the endometriosis is superficial endometriosis (stage I/II) or deep infiltrating endometriosis (stage III/IV).

12. The method of claim 1, wherein the endometrial stromal cells are isolated by sorting the cells using endometrial stromal cell markers CD10, CD146, and CD13.

13. The method of claim 2, wherein the endometrial epithelial cells are isolated by sorting the cells using endothelial epithelial cell markers EpCam+, CD45, and CD9.

14. The method of claim 1, wherein the one or more genes whose expression is reduced relative to expression of the one or more genes in endometrial stromal cells from a subject without endometriosis include Cx43, MAPK, TGFBR2, ZO2 and ZO1.

15. The method of claim 1, wherein the one or more genes whose expression is reduced relative to expression of the one or more genes in endometrial stromal cells from a subject without endometriosis include SNAIl, Twist 1 Zeb2, Notch1, VEGFR1 and CD45.
